(19) United States
(12) Patent Application Publication
(10) Pub. No.: US 2005/0178584 A1
(43) Pub. Date: Aug. 18, 2005

(54) Coated stent and MR imaging thereof

(76) Inventors: Xingwu Wang, Wellsville, NY (US); Robert W. Gray, Rochester, NY (US); Jeffrey L. Helfer, Victor, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 11/052,263

(22) Filed: Feb. 7, 2005

Related U.S. Application Data
(63) Continuation-in-part of application No. 10/090,553, filed on Mar. 4, 2002.
Application No. 10/090,553 is a continuation-in-part of application No. 10/054,407, filed on Jan. 22, 2002, now Pat. No. 6,506,972.
(63) Continuation-in-part of application No. 10/810,916, filed on Mar. 26, 2004, now Pat. No. 6,846,985.
(63) Continuation-in-part of application No. 10/808,618, filed on Mar. 24, 2004.
(63) Continuation-in-part of application No. 10/786,198, filed on Feb. 25, 2004.
(63) Continuation-in-part of application No. 10/780,045, filed on Feb. 17, 2004.
(63) Continuation-in-part of application No. 10/747,472, filed on Dec. 29, 2003.
(63) Continuation-in-part of application No. 10/744,543, filed on Dec. 22, 2003.
(63) Continuation-in-part of application No. 10/442,420, filed on May 21, 2003, now Pat. No. 6,914,412.
(63) Continuation-in-part of application No. 10/409,505, filed on Apr. 8, 2003, now Pat. No. 6,815,609.
(60) Provisional application No. 60/542,270, filed on Feb. 5, 2004.

(51) Int. Cl. A61F 2/06

(52) U.S. Cl. 174/256

(57) ABSTRACT

Disclosed in this specification is a stent coated with a layer comprised of particulates which have an average particle size of less than 100 nanometers; a saturation magnetization of at least 2,000 gauss; and where the average coherence length between the particulates is from about 1 nanometer to about 50 nanometers.

### CROSS-REFERENCE TO RELATED PATENT APPLICATIONS

This application is a continuation-in-part of applicants' co-pending patent application U.S. Ser. No. 10/090,553 filed on Mar. 4, 2002 which is a continuation-in-part of patent application U.S. Ser. No. 10/054,407 filed on Jan. 22, 2002, now U.S. Pat. No. 6,506,972. This application also claims the benefit of the filing date of U.S. provisional patent application U.S. Ser. No. 60/542,270 filed Feb. 5, 2004. This application is also a continuation-in-part of each of applicants' co-pending patent application Ser. No. 10/810,916 (filed on Mar. 26, 2004), Ser. No. 10/808,618 (filed on Mar. 24, 2004), Ser. No. 10/786,198 (filed on Feb. 25, 2004), Ser. No. 10/780,045 (filed on Feb. 17, 2004), Ser. No. 10/747,472 (filed on Dec. 29, 2003), Ser. No. 10/744,543 (filed on Dec. 22, 2003), Ser. No. 10/442,420 (filed on May 21, 2003), and Ser. No. 10/409,505 (filed on Apr. 8, 2003). The entire contents of the above referenced patents and patent applications are hereby incorporated by reference into this specification.

### FIELD OF THE INVENTION

This invention relates, in one embodiment, to a prosthesis adapted to be visualized under magnetic resonance imaging (MRI) conditions and more particularly to a stent adapted to permit visualization of plaque under MRI conditions.

### BACKGROUND OF THE INVENTION

Medical stents are widely used to treat obstructed lumens, such as blood vessels. Stents are surgically implanted within the lumen of a biological organism. Over time, the undesired reocclusion of the lumen occurs as plaque forms on the surface of the stent; a process referred to as restenosis.

Numerous attempts to address restenosis may be found in the prior art. These attempts have been thwarted by an inability to easily detect plaque formation. It would be desirable to visualize plaque within a stent by non-invasive means, such as magnetic resonance imaging (MRI).

U.S. Pat. No. 6,767,360 to Alt (Vascular Stent with Composite Structure for Magnetic Resonance Imaging Capabilities) teaches “a stent is adapted to be implanted in a duct of a human body to maintain an open lumen at the implant site, and to allow viewing body tissue and fluids by magnetic resonance imaging (MRI) energy applied external to the body. The stent constitutes a metal scaffold. An electrical circuit resonant at the resonance frequency of the MRI energy is fabricated integral with the scaffold structure of the stent to promote viewing body properties within the lumen of the stent.” As recited in column 1 of this patent, “A drawback of stenting is the body's natural defensive reaction to the implant of a foreign object. In many patients, the reaction is characterized by a traumatic proliferation of tissue as intimal hyperplasia at the implant site, and, where the stent is implanted in a blood vessel such as a coronary artery, formation of thrombi which becomes attached to the stent. Each of these adverse effects contributes to restenosis—a re-narrowing of the vessel lumen—to compromise the improvements that resulted from the initial re-opening of the lumen by implanting the stent. Consequently, a great number of stent implant patients must undergo another angiogram, on average about six months after the original implant procedure, to determine the status of the tissue proliferation and thrombosis in the affected lumen. If re-narrowing has occurred, one or more additional procedures are required to stem or reverse its advancement. For virtually all stent implant patients it is desirable to examine and analyze the patency of the vessel lumen and the extent of tissue growth within the lumen of the stent, and to measure blood flow therethrough, from time to time as part of the patient's routine post-procedure examinations. Current techniques employed to analyze patency of the lumen following a stent implant procedure are more or less invasive.”

U.S. Pat. No. 6,280,385 to Meizer (Stent and MR Imaging Process for the Imaging and the Determination of the Position of a Stent) teaches a stent that has “ . . . at least one passive resonance circuit with an inductance and a capacitance whereby its resonance frequency is essentially equal to the resonance frequency of the applied high-frequency radiation of the magnetic resonance system.” The stent has improved visibility relative to prior art stents.

U.S. Pat. No. 6,606,513 to Lardo (Magnetic Resonance Imaging Transseptal Needle Antenna) discloses “ . . . an MRI transseptal needle that can be visible on an MRI, can act as an antenna and receive MRI signals from surrounding subject matter to generate high-resolution images and can enable real-time active needle tracking during MRI guided transseptal puncture procedures.”

U.S. Pat. Nos. 6,799,067 and 6,845,259 to Pacetti (MRI Compatible Guide Wire) disclose “ . . . a guide wire or other guiding member for use within a patient's body that is at least in part visible under magnetic resonance imaging (MRI) but is not detrimentally affected by the imaging.” Reference may also be had to U.S. Pat. No. 6,712,844 to Pacetti (MRI Compatible Stent); U.S. Pat. No. 6,585,755 to Jackson (Polymeric Stent Suitable for Imaging by MRI and Fluoroscopy); and U.S. Pat. No. 6,574,497 to Pacetti (MRI Medical Device Markers Utilizing Fluorine-19).

U.S. Pat. No. 6,786,904 to Döscher (Method and Device to Treat Vulnerable Plaque) discloses a stent-like-structure that is adapted to remove plaque by heat ablation.

U.S. Pat. No. 6,802,857 to Walsh (MRI Stent) teaches a “ . . . stent device [that] includes an electrically conductive helical structure. The stent device also includes an electrically conductive ring structure connected to the helical structure. The ring structure includes an inner conducting ring, an outer conducting ring, and a dielectric material disposed between the inner and outer conducting rings. The helical structure and the ring structure are arranged to produce an electromagnetic field when subjected to an applied electromagnetic field.”

U.S. Pat. No. 6,831,644 to Lienard (Method and Device for Displaying the Deployment of an Endovascular Prosthesis) discloses means for obtaining magnified images of a stent “ . . . images of which are acquired by means of a radiography machine of the type comprising an X-ray source and an image detector and an image display placed opposite the source.”

U.S. patent application 2004/0225326 to Weiner (Apparatus for the Detection of Restenosis) teaches a stent adapted to detect plaque within the lumen of the stent using electromagnetic radiation. Other attempts to detect or treat restenosis include U.S. Pat. Nos. 6,015,387; 6,170,488; 6,200,307; 6,488,704; 6,491,666 and 6,656,162. The contents of U.S. Pat. Nos. 6,015,387; 6,170,488; 6,200,307; 6,280,385; 6,488,704; 6,491,666; 6,574,497; 6,585,755; 6,606,513; 6,656,162; 6,712,844; 6,767,360; 6,786,904; 6,799,067; 6,802,857; 6,831,644; 6,845,259; and U.S. patent application 2004/0225326 are hereby incorporated by reference into this specification.

As disclosed in U.S. Pat. No. 6,786,904 “Today's conventional stents are visible under MRI. Stents made of stainless steel show a rather large image distortion or a blur of the image, referred to as image artifact. This image artifact is created by a local distortion of the MR magnetic field conditions due to the magnetic susceptibility of the stent material used.” The aforementioned image artifact greatly complicates the detection of restenosis in stents under MRI conditions.

It is an object of this invention to provide a stent that more easily allows for the detection of restenosis under MRI conditions than do prior art stents.

### SUMMARY OF THE INVENTION

In accordance with this invention, there is provided a coated stent adapted to be more easily visualized under MR imaging conditions. The techniques and materials described in this specification are advantageous because they are more simple compared to other prior art approaches and may be adapted to function with a variety of stents.

### BRIEF DESCRIPTION OF THE DRAWINGS

The invention will be described by reference to the following drawings, in which like numerals refer to like elements, and in which:

FIG. 1 is a perspective view of a stent disposed within a lumen;

FIGS. 2A, 2B and 2C are end views of three stents;

FIG. 3 is a schematic diagram of a certain circuit;

FIG. 4 is a phase diagram of one composition of the invention;

FIGS. 5A, 5B and 6 are perspective views of coated substrates of the present invention;

FIG. 7 is a flow diagram of one process of the present invention;

FIG. 8 is an illustration of two out of phase waves used in the present invention; and

FIG. 9 is a photograph of various stents under magnetic resonance imaging conditions both before and after digital post-processing.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims. Specifically, the preferred embodiment described herein is a coated stent, but it should be understood that other articles of manufacture may be likewise coated.

### DESCRIPTION OF THE PREFERRED EMBODIMENTS

In accordance with this invention, there is provided a coated stent adapted to be more easily visualized under MR imaging conditions. For a general understanding of the present invention, reference is made to the drawings. In the drawings, like reference numerals have been used throughout to designate identical elements.

Applicants have discovered that certain stents, when coated with certain particles, allow for easier visualization of objects contained within the lumens of the stents (for example, plaque due to restenosis). Magnetic resonance imaging can be used to visualize features within a biological organism if there is no magnetic resonance distortion. Many prior art stents give rise to such a distortion, and thus interfere with MR imaging of stents. Without wishing to be bound to any particular theory, applicants believe that the ability to image inside a stent is determined by the super-position of a plurality of factors including (1) eddy currents induced by (a) inductive coupling (Faraday's law): a conductive loop in a changing magnetic field will develop an EMF (b) surface boundary conditions for a conductor and an RF (radio frequency) wave; (2) magnetic susceptibility of the stent materials; (3) dielectric effects: body tissues and fluids near the stent have different electrical properties (4) geometry of the stent and (5) alignment of the stent to the magnetic field of the MR scanner. Applicants believe that the challenge of imaging within a stent is two-fold.

First, it would be advantageous to allow the MR transmit signal to penetrate the interior of the stent without distortion. Second, it would also be advantageous to allow the MR signal from the tissues within the stent to leave the stent without distortion and to be picked up by the MR receive coils. Nullification of the effects of stent substrate magnetization and reduction in MR-induced eddy and loop currents would help achieve both of these goals. Creation of a controllable phase shift, combined with enhanced edge discrimination, would help to produce a more reliable MR image.

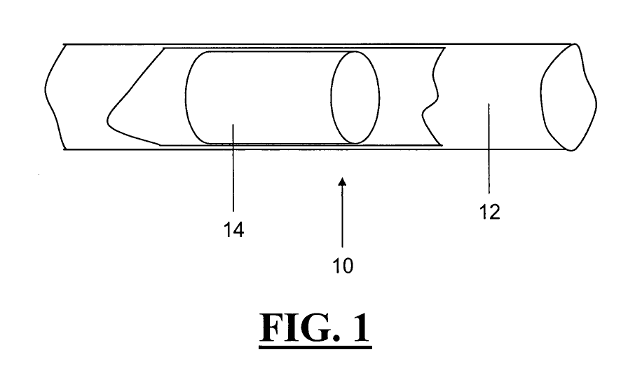

FIG. 1 depicts apparatus 10 wherein stent 14 is disposed within lumen 12. In the embodiment depicted in FIG. 1, lumen 12 is a blood vessel. For example, lumen 12 may be an artery or vein. As would be apparent to one skilled in the art, other lumens may be used, such as genitourinary lumens and the like. In one embodiment stent 12 is comprised of nitinol. Reference may be had to U.S. Pat. No. 5,147,370 to McNamara (Nitinol Stent for Hollow Body Conduits). In another embodiment, stent 12 is comprised of copper. Reference may be had to U.S. Pat. No. 4,969,458 to Wiktor (Intracoronary stent and method of simultaneous angioplasty and stent implant). The contents of U.S. Pat. Nos. 4,969,458 and 5,147,370 are hereby incorporated by reference into this specification. Such stents are inexpensive, but cause image artifacts when subjected to MRI conditions. These image artifacts make visualization of plaque within the stent difficult.

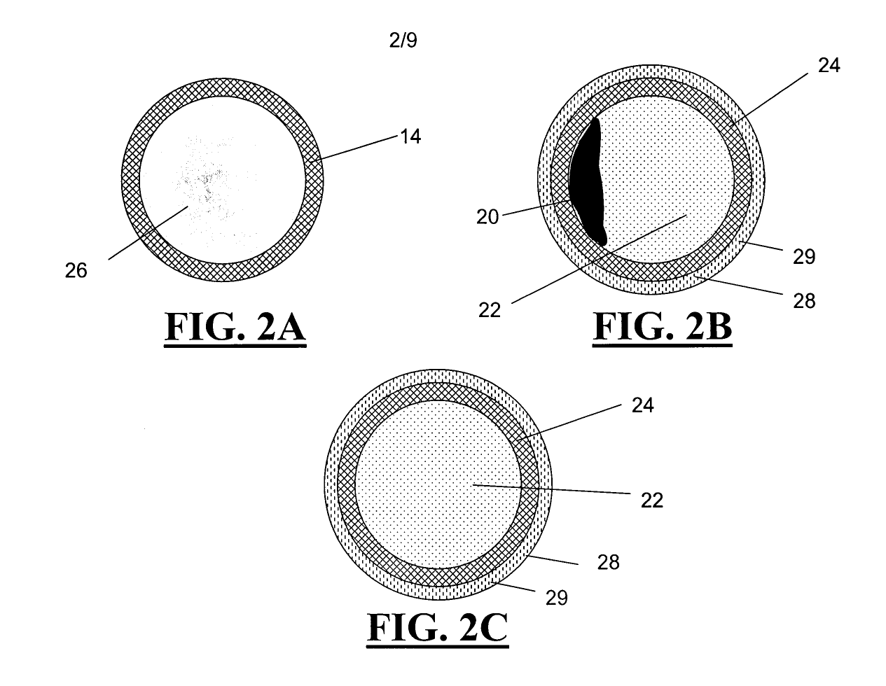

FIG. 2A is an end view of stent 14 under MR imaging conditions. As can be seen in FIG. 2A, image artifact 26 obscures any plaque that may be present in stent 14.

FIG. 2B is an end view of coated stent 24. Disposed about stent 24 is a layer 28 of particles 29. These particles are adapted to at least partially correct the image artifact so that plaque 20 can be visualized within lumen 22 under MRI conditions. In the embodiment depicted in FIG. 2B, plaque 20 is present. In the embodiment depicted in FIG. 2C, no plaque was detected. Applicants believe that the layer of particles alters the electronic properties of the stent and allows for visualization of the plaque.

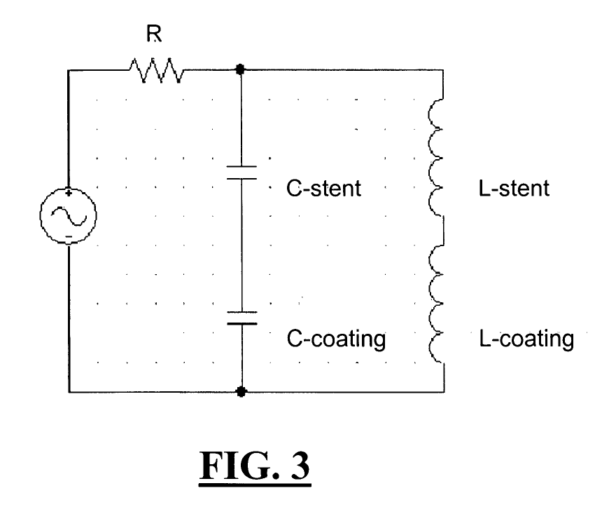

FIG. 3 is a symbolic representation of a circuit diagram that illustrates one theory regarding the operation of the present invention. Without wishing to be bound to any particular theory, applicants believe the stent in the MR scanner functions as an electrical circuit. FIG. 3 is an approximation and simplification to the equivalent circuit for the stent plus coatings.

As illustrated in FIG. 3, the electrical properties of the coated stents are tunable. As illustrated in FIG. 3, the coating provides an additional inductance (“L-coating”) to the stent. The dielectric properties of the coatings provide an additional capacitance (“C-coating”) to the stent. This allows the stent-coating-tissue system's “circuit” parameters to be adjusted by varying the composition of the coating. This adjustment capability appears to provide several advantages: (1) the phase characteristics of the MR signal emitting from the tissue may be controllably adjusted and discriminated in the phase data; (2) nullification of field inhomogeneities due to the MR-induced magnetization of stent materials; (3) reduction of MR-induced eddy and loop currents by changing the surface resistance and the impedance characteristics of the stents, and (4) enhanced edge discrimination compared to uncoated stents.

The nullification of field inhomogeneities may be accomplished by altering the composition of the particles (such as iron content) and layer properties (such as thickness). This coating adjustment is interrelated to other stent/coating properties. For example, stents may be made from a variety of materials, including copper and nitinol. Copper has a magnetic susceptibility less than zero while nitinol has a magnetic susceptibility greater than zero. Therefore, the present coatings need to be adjusted differently depending on the substrate that the coatings are applied to. The inductance of the system is related to magnetic susceptibility. The capacitance is related to the dielectric properties. In the case of a nitinol stent, adjustment of the coating capacitance in this oscillating system combined with the inductance properties helps to reduce some of the magnetic susceptibility effects of the substrate.

Applicants have discovered a certain particulate that, when coated onto the surface of a stent, enhances the image of the stent under certain MRI conditions. Such materials have been previously taught in other patents. Reference may be had to the following United States patents to Wang: U.S. Pat. No. 6,506,972 (Magnetically Shielded Conductor); U.S. Pat. No. 6,673,999 (Magnetically Shielded Assembly); U.S. Pat. No. 6,713,671 (Magnetically shielded Assembly); U.S. Pat. No. 6,765,144 (Magnetic Resonance Imaging Coated Assembly); U.S. Pat. No. 6,815,609 (Nanomagnetic Composition); U.S. Pat. No. 6,844,492 (Magnetically Shielded Conductor); and U.S. Pat. No. 6,846,985 (Magnetically Shielded Assembly). For information related to the general state of the art, reference may be had to U.S. Pat. No. 6,225,565 to Prysner and U.S. Pat. No. 5,927,621 to Ziolo. Other particles are similarly disclosed in U.S. Pat. Nos. 5,889,091; 5,714,136; 5,667,924; and 5,213,851. The contents of the aforementioned patents are hereby incorporated by reference into this specification.

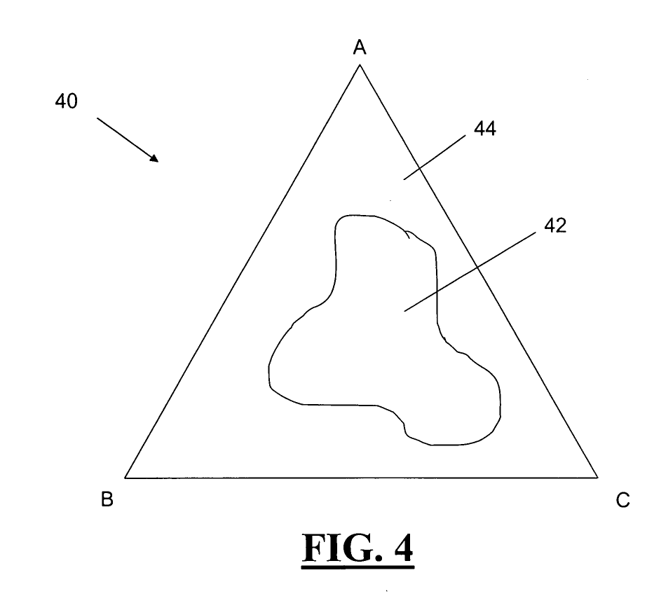

Generally, the particles of the present invention are comprised of three moieties, denoted A, B, and C. In one embodiment, the particles of this invention are comprised of aluminum, iron, and nitrogen atoms. In another embodiment, the particles are comprised of aluminum, iron, and a mixture of nitrogen and oxygen atoms. These embodiments are illustrated in FIG. 4.

FIG. 4 illustrates a phase diagram comprised of moieties A, B, and C. A is a magnetic moiety. In one embodiment, moiety A is selected from the group consisting of iron, nickel, samarium, and gadolinium. In another embodiment, moiety A is selected from the group consisting of iron and nickel. In yet another embodiment, moiety A is iron.

Referring again to FIG. 4, it is preferred that the moiety B have a resistivity of from about 2 to about 100 microohm-centimeters. In one embodiment, moiety B is selected from the group consisting of aluminum, copper, gold, silver, and mixtures thereof. In one embodiment, B is aluminum with a resistivity of about 2.824 microohm-centimeters. As will apparent, other materials with resistivities within the desired range also may be used. In one embodiment, B is selected from the group consisting of aluminum, silicon, copper, and combinations thereof. In another embodiment, B is selected from the group consisting of aluminum, silicon and copper. In another embodiment, B is selected from the group consisting of aluminum and copper. In yet another embodiment, B is aluminum. As would be apparent to one skilled in the art, other similar elements may also be used.

Referring again to FIG. 4, in one embodiment, C is selected from the group consisting of nitrogen, oxygen, carbon and combinations thereof. In another embodiment, C is selected from the group consisting of nitrogen, oxygen, and combinations thereof. In yet another embodiment C is a mixture of oxygen and nitrogen.

Without wishing to be bound to any particular theory, it is believed that the particles found in these coatings have very small magnetic domains (in one embodiment from about 3 to about 10 nanometers) as opposed to the very large “bulk” magnetic domains in other, prior art magnetic materials. This causes the magnetic moments of the coatings to respond to external field changes. Additionally, the particles may be dispersed within a matrix. When the particles are disposed within a matrix the resulting coating may allow more magnetic flux lines to pass through the coating and into the stent's interior than traditional bulk magnetic materials would. In one embodiment, this matrix is comprised of aluminum and nitrogen. The size of the magnetic domains can be related to the coherence length between the particles. The coherence length between adjacent A moieties is, on average, preferably from about 0.1 to about 100 nanometers and, more preferably, from about 1 to about 50 nanometers. In one embodiment, the coherence length is from about 3 to about 20 nanometers.

Without wishing to be bound to any particular theory, applicants believe the small magnetic domain is a result of, at least in part, the small size of the particles. In one embodiment, the average particle size of the particulates is less than 100 nanometers. In another embodiment, the average particle size of the particulates is less than about 50 nanometers. In another embodiment, the average particle size of the particulates is from about 2 nanometers to about 50 nanometers. In another embodiment, the average particle size of the particulates is from about 2 nanometers to about 10 nanometers.

In one embodiment of the invention, the particulate material is coated onto a stent to form a layer so as to provide a saturation magnetization, at 25 degrees centigrade, of a certain value. In one embodiment, this layer has a saturation magnetization of at least about 2,000 gauss. In another embodiment, the layer of particulate material has a saturation magnetization of at least about 5,000 gauss. In another embodiment, the layer of particulate material has a saturation magnetization of at least about 10,000 gauss. In another embodiment, the layer of particulate material has a saturation magnetization of at least about 20,000 gauss. In yet another embodiment, the layer of particulate material has a saturation magnetization of at least about 26,000 gauss. For a discussion of saturation magnetization, reference may be had to U.S. Pat. Nos. 4,705,613; 4,631,613; 5,543,070; 3,901,741 and the like. The contents of these patents are hereby incorporated by reference into this specification. As will be apparent to those skilled in the art, especially upon studying the aforementioned patents, the saturation magnetization of thin films is often higher than the saturation magnetization of bulk objects.

The aforementioned layer of particulate material may be coated onto the stent in various thicknesses. In one embodiment, the thickness of the layer is less than about 100 microns. In one embodiment, the thickness of the layer is less than about 10 microns. In another embodiment, the thickness of the layer is less than about 5 microns. In another embodiment, the thickness of the layer is from about 0.1 to about 3 microns. The thickness of the layer of particulate material is measured from the bottom surface of the layer that contains such material to the top surface of such layer that contains such material; and such bottom surface and/or such top surface may be contiguous with other layers of material (such as insulating material) that do not contain these particles. In one embodiment, these other layers consist essentially of aluminum and nitrogen.

Methods for coating surfaces with such particles are well known to those skilled in the art. Reference may be had to U.S. Pat. No. 6,398,806 to You (Monolayer Modification to Gold Coated Stents to Reduce Adsorption of Protein). The coated stents of this invention may be prepared by other conventional means such as, e.g., the process described in U.S. Pat. No. 5,540,959 to Wang (Process for Preparing a Coated Substrate). This patent describes and claims a process for preparing a coated substrate, comprising the steps of: (a) creating mist particles from a liquid, wherein: 1. said liquid is selected from the group consisting of a solution, a slurry, and mixtures thereof, 2. said liquid is comprised of solvent and from 0.1 to 75 grams of solid material per liter of solvent, 3. at least 95 volume percent of said mist particles have a maximum dimension less than 100 microns, and 4. said mist particles are created from said first liquid at a rate of from 0.1 to 30 milliliters of liquid per minute; (b) contacting said mist particles with a carrier gas at a pressure of from 761 to 810 millimeters of mercury; (c) thereafter contacting said mist particles with alternating current radio frequency energy with a frequency of at least 1 megahertz and a power of at least 3 kilowatts while heating said mist particles to a temperature of at least about 100 degrees centigrade, thereby producing a heated vapor; (d) depositing said heated vapor onto a substrate, thereby producing a coated substrate; and (e) subjecting said coated substrate to a temperature of from about 450 to about 1,400 degrees centigrade for at least about 10 minutes. The contents of U.S. Pat. Nos. 5,364,562; 5,540,959 and 6,398,806 are hereby incorporated by reference into this specification.

As discussed elsewhere in this specification, coatings of certain particles can be applied to the stent so as to alter some of the stent's electrical parameters. These parameters include the stent's surface resistance and its overall inductance. Additionally, the coatings provide a dielectric layer between the conductor of the stent and the body tissue and fluids. This enables multilayer coatings to form a capacitance per unit length of the stent. In one embodiment, one of these layers is a matrix that consists essentially of aluminum and nitrogen.

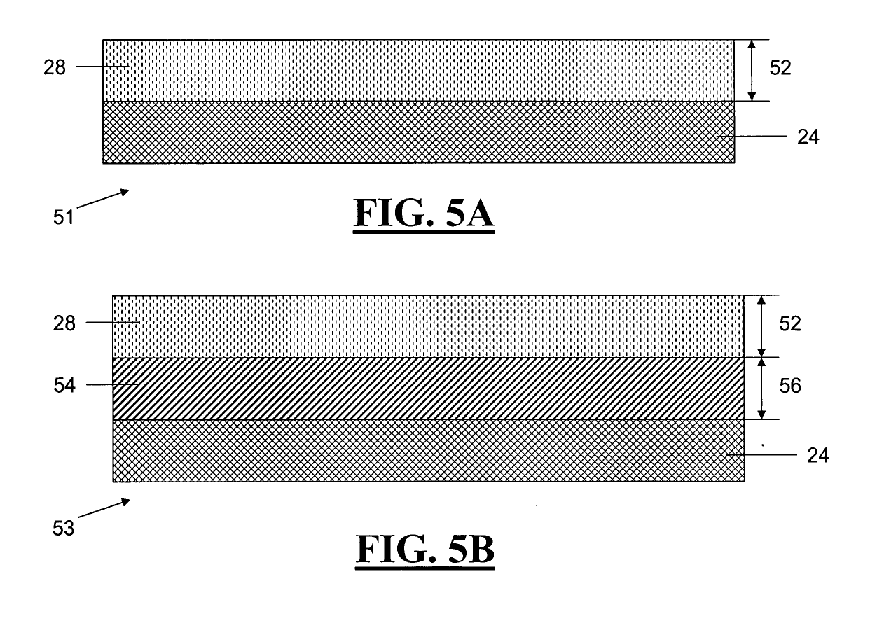

FIG. 5A depicts a single layered coating assembly 51. In the embodiment depicted in FIG. 5A, stent 24 is coated with layer 28 with a thickness 52. The particles (not shown) of the present invention are disposed within layer 28. As would be apparent to one skilled in the art, the stents are often coating on all sides. For the sake of clarity, only one side is shown as coated.

FIG. 5B illustrates a multilayered coating assembly 53. In the embodiment depicted in FIG. 5B, stent 24 is coated with layer 54 which had a thickness 56. In one embodiment, layer 54 consists essentially of aluminum and nitrogen. Disposed above layer 54, and congruent therewith, is layer 28. The particles (not shown) of the present invention are disposed within layer 28.

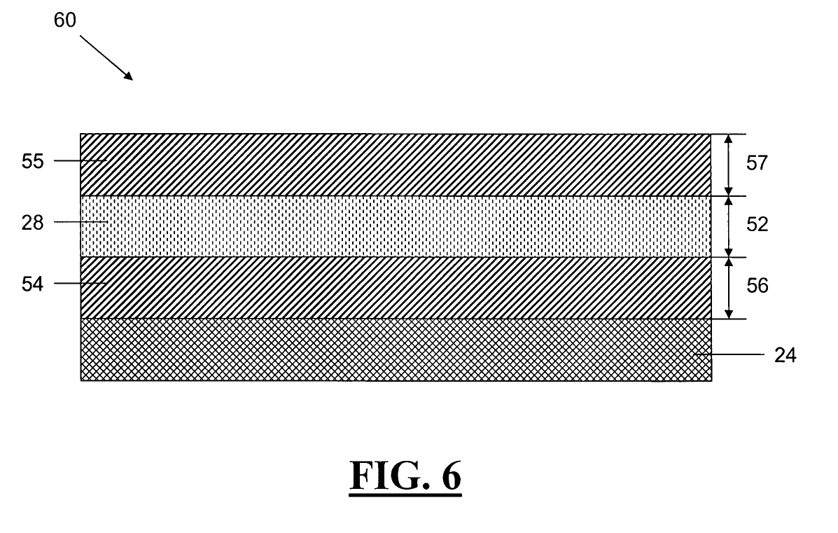

FIG. 6 is an illustration of another multilayered coating assembly 60. In the embodiment depicted in FIG. 6, stent 24 is coated with layer 54 which has a thickness 56. In one embodiment, layer 54 consists essentially of aluminum and nitrogen. Disposed above layer 54, and contiguous therewith, is layer 28 which has a thickness 52. Disposed within layer 28 are the particles (not shown) of the invention. Disposed above layer 28 is layer 55. In one embodiment, layer 55 is comprised of substantially the same material as layer 54. In the embodiment depicted in FIG. 6, layers 28, 54 and 55 have approximately the same thickness. In other embodiments (not shown) the thicknesses may vary.

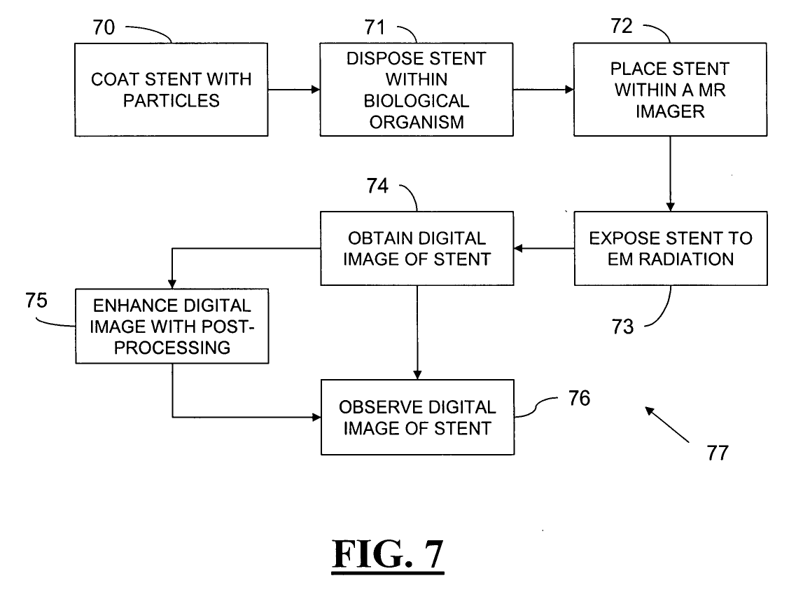

FIG. 7 is a flow diagram of one process 77 of the invention. In process 77, a coated stent is imaged by MRI. In step 70, a stent is coated with particles in accordance with the teachings of this specification. In step 71, which is optional, the coated stent is disposed with a biological organism. In one embodiment, the biological organism is a human being. In step 72 of the process, the coated stent is placed within a MR imaging scanner. In step 73 the stent is exposed to electromagnetic radiation (for example, radio frequency waves) from the MR imaging scanner. In step 74, a digital image of the stent is produced by the MR imaging scanner. In step 76, the resulting digital image is observed by the end user. In this manner, the end user can visualize the interior of the stent and examine it for plaque formation. In step 75, which is optional, the digital image is enhanced with post-processing software prior to being observed.

A variety of suitable post-processing techniques are known to those skilled in the art. It is well established that digital image provided by MR imaging may be separated into magnitude data and phase data. Digital imaging software may be used to enhance the magnitude and phase data.

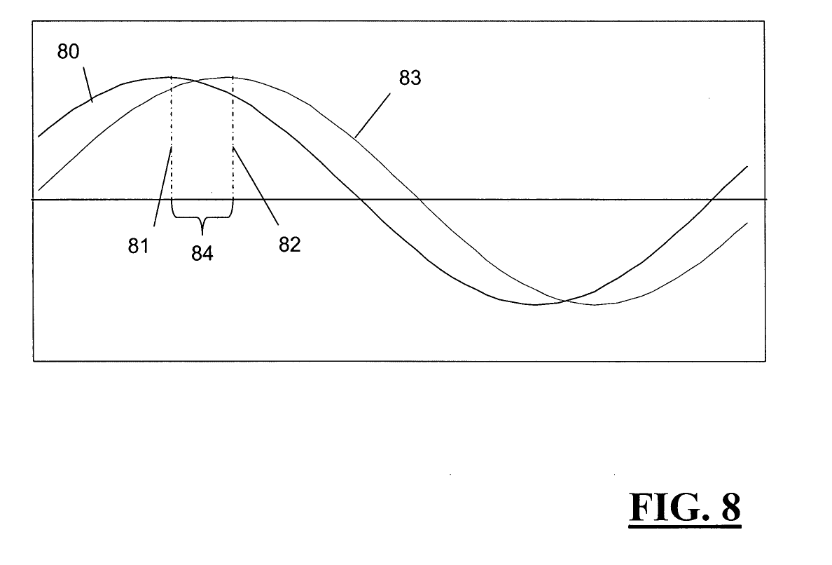

FIG. 8 is an illustration of two electromagnetic waves; wave 80 and wave 83. Waves 80 and 83 have magnitudes 81 and 82 respectively. Waves 80 and 83 are out of phase in time by phase difference 84. Prior art digital image process methods teach the use of magnitude and phase data to perform enhancements of digital images. Reference may be had to U.S. Pat. No. 6,674,904 to McQueen (Contour Tracing and Boundary Detection for Object Identification in a Digital Image); U.S. Pat. No. 6,215,983 to Dogan (Method and Apparatus for Comlex Phase Equalization for use in a Communication System); U.S. Pat. No. 6,370,224 to Simon (System and Methods for the Reduction and Elimination of Image Artifacts in the Calibration of X-Ray Imagers); U.S. Pat. No. 6,011,862 Doi (Computer-Aided Method for Automated Image Feature Analysis and Diagnosis of Digitized Medical Images); U.S. Pat. No. 6,007,052 to Tinkler (System and Method for Local Area Image Processing); U.S. Pat. No. 6,005,983 to Anderson (Image Enhancement by Non-Linear Extrapolation in Frequency Space); U.S. Pat. No. 5,790,690 to Doi (Computer-Aided Method for Automated Image Feature Analysis and Diagnosis of Medical Images); U.S. Pat. No. 5,717,789 to Anderson (Image Enhancement by Non-Linear Extrapolation in Frequency Space); U.S. Pat. No. 5,621,802 to Harjani (Apparatus for Eliminating Acoustic Oscillation in a Hearing Aid Using Phase Equalization); U.S. Pat. No. 5,598,302 to Park (Method and Apparatus for Detecting Digital Playback Signals using Phase Equalization and Waveform Shaping of Playback Signals); U.S. Pat. No. 5,325,317 to Petersen (Digitally Programmable Linear Phase Filter Having Phase Equalization); U.S. Pat. No. 5,122,788 TO Sid-Ahmed (Method and an Apparatus for 2-D Filtering a Raster Scanned Image in Real-Time); U.S. Pat. No. 5,061,925 to Sooch (Phase Equalization System for a Digital-to-Analog Converter Utilizing Separate Digital and Analog Sections); and U.S. Pat. No. 4,771,398 to Brandstetter (Method and Apparatus for Optical RF Phase Equalization). The contents of the aforementioned patents are hereby incorporated by reference into this specification.

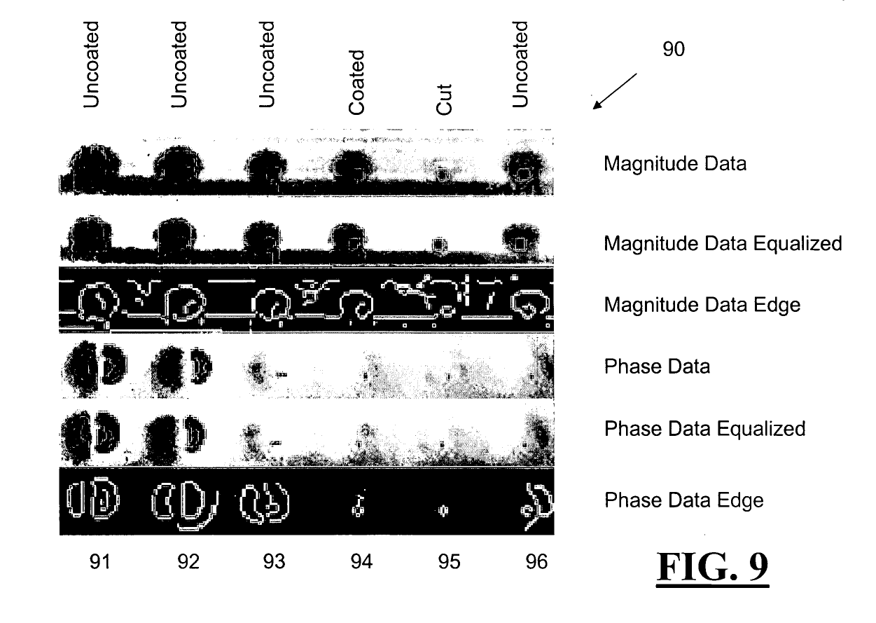

FIG. 9 is a photograph 90 of six copper rings under MR imaging conditions. A nylon rod was disposed within the rings to simulate an object within a stent. The first row is the MR image wherein the image was constructed using the magnitude data from the MR scanner. The second and thirds rows are the same image after post-processing enhancements have been applied. In the second row magnitude equalization was used to enhance the image. In the third row the magnitude data was used to perform an edge detection algorithm. The fourth row is the MR image wherein the image was constructed using the phase data from the MR scanner. The fifth and sixth rows are the same image after post-processing enhancements have been applied. In the fifth row, phase equalization was used to enhance the image. In the sixth row the phase data was used to perform an edge detection algorithm.

Referring again to FIG. 9, six copper rings, 91, 92, 93, 94, 95, and 96 are illustrated. Ring 95 has been cut so that it functions as an idealized reference. Since ring 95 is cut, it has no eddy currents. Thus, the nylon rod within ring 95 is clearly visible under MRI conditions. For example, when an edge detection algorithm is run on the phase data (row six) the nylon rod is quite apparent. Rings 91, 92, and 93 are uncoated copper rings which serve as a reference. It is clear from FIG. 9 that the nylon rod within rings 91, 92 and 93 are much more difficult to detect. In contrast, ring 94 is coated in accordance with the teachings of this invention. The nylon object within ring 94 is visible under MR imaging conditions. In particular, the appearance of the nylon object within ring 94 is markedly superior to the non-coated rings 91 to 93. This is especially true after the image has been subjected to a phase edge detection algorithm. The phase edge detection of ring 94 is substantially to the idealized cut ring 95.

It is therefore, apparent that there has been provided, in accordance with the present invention, a method and apparatus for the detection of restenosis within the lumen of a stent by MR imaging. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A coated substrate comprising a substrate coated with a layer comprised of particulates wherein
a. said particulates have an average particle size of less than about 100 nanometers; and
b. said layer has a saturation magnetization of at least about 2,000 gauss.

2. The coated substrate as recited in claim 1, wherein said layer has a thickness of less than about 100 microns.

3. The coated substrate as recited in claim 2, wherein said saturation magnetization is at least about 5,000 gauss.

4. The coated substrate as recited in claim 3, wherein said saturation magnetization is at least about 10,000 gauss.

5. The coated substrate as recited in claim 4, wherein said saturation magnetization is at least about 20,000 gauss.

6. The coated substrate as recited in claim 3, wherein said thickness is less than about 10 microns.

7. The coated substrate as recited in claim 6, wherein said substrate is a stent.

8. The coated substrate as recited in claim 7, wherein said stent is selected from the group consisting of a nitinol stent and a copper stent.

9. The coated substrate as recited in claim 7, wherein said particulates have an average particle size of less than 50 nanometers.

10. The coated substrate as recited in claim 9, wherein said particulates have an average particle size of from about 2 nanometers to about 50 nanometers.

11. The coated substrate as recited in claim 10, wherein said particulates have an average particle size of about 2 nanometers to about 10 nanometers.

12. The coated substrate as recited in claim 1 wherein the average coherence length between said particulates is from about 0.1 nanometers to about 100 nanometers.

13. A coated substrate comprising a stent coated with a layer comprised of particulates wherein
a. said particulates have an average particle size of less than 100 nanometers;
b. said layer has a saturation magnetization of at least 2,000 gauss;
c. the average coherence length between said particulates is from about 1 nanometer to about 50 nanometers.

14. The coated substrate as recited in claim 2, wherein said saturation magnetization is at least about 5,000 gauss.

15. The coated substrate as recited in claim 3, wherein said saturation magnetization is at least about 10,000 gauss.

16. The coated substrate as recited in claim 4, wherein said saturation magnetization is at least about 20,000 gauss.

17. A coated substrate comprising a stent coated with a first layer comprised of particulates wherein
a. said particulates have an average particle size of less than about 100 nanometers;
b. said first layer has a saturation magnetization of at least about 2,000 gauss;
c. said particulates are comprised of a first component, a second component, and a third component wherein
i. said first component is selected from the group consisting of iron, nickel, samarium, and gadolinium;
ii. said second component is selected from the group consisting of aluminum, silicon, copper, and combinations thereof; and
iii. said third component is selected from the group consisting of nitrogen, oxygen, carbon and combinations thereof.

18. The coated substrate as recited in claim 17, wherein said saturation magnetization is at least about 5,000 gauss.

19. The coated substrate as recited in claim 18, wherein said saturation magnetization is at least about 10,000 gauss.

20. The coated substrate as recited in claim 19, wherein said saturation magnetization is at least about 20,000 gauss.

21. The coated substrate as recited in claim 17, wherein
a. said first component is selected from the group consisting of iron, nickel, and combinations thereof;
b. said second component is selected from the group consisting of aluminum and copper, and combinations thereof; and
c. said third component is selected from the group consisting of nitrogen, oxygen, and combinations thereof.

22. The coated substrate as recited in claim 17, wherein
a. said first component is selected from the group consisting of iron, nickel;
b. said second component is selected from the group consisting of aluminum and copper; and
c. said third component is selected from the group consisting of nitrogen, oxygen, and combinations thereof.

23. The coated substrate as recited in claim 22, wherein said first layer is further comprised of a matrix wherein said particles are disposed within said matrix.

24. The coated substrate as recited in claim 23, wherein said matrix is comprised of aluminum and nitrogen.

25. The coated substrate as recited in claim 22, wherein said first layer is congruent with said stent.

26. The coated substrate as recited in claim 22, wherein said coated substrate is further comprised of a second layer which consists essentially of aluminum and nitrogen.

27. The coated substrate as recited in claim 26, wherein said second layer is disposed between said first layer and said stent.

28. The coated substrate as recited in claim 27, wherein said coated substrate is further comprised of a third layer which consists essentially of aluminum and nitrogen.

29. The coated substrate as recited in claim 28, wherein said first layer is disposed between said second layer and said third layer.

30. The coated substrate as recited in claim 22, wherein
a. said first component is iron;
b. said second component is aluminum;
c. said third component is selected from the group consisting of nitrogen, oxygen, and combinations thereof.

31. The coated substrate as recited in claim 21, wherein said first component is present in said first layer in a concentration from about 1% to about 40% by weight by total weight of said first component and said second component.

32. The coated substrate as recited in claim 31, wherein said first component is present in said first layer in a concentration from about 1% to about 30% by weight by total weight of said first component and said second component.

33. The coated substrate as recited in claim 32, wherein said first component is present in said first layer in a concentration from about 1% to about 20% by weight by total weight of said first component and said second component.

34. The coated substrate as recited in claim 33, wherein said first component is present in said first layer in a concentration from about 5% to about 15% by weight by total weight of said first component and said second component.

35. A coated substrate comprising a stent coated with a first layer comprised of particulates wherein
a. said particulates have an average particle size of less than about 100 nanometers;
b. said first layer has a saturation magnetization of at least about 2,000 gauss;
c. said particulates consist essentially of a first component, a second component, and a third component wherein
i. said first component is selected from the group consisting of iron, nickel, samarium, and gadolinium;
ii. said second component is selected from the group consisting of aluminum, silicon, copper, and combinations thereof; and
iii. said third component is selected from the group consisting of nitrogen, oxygen, carbon and combinations thereof.

36. A process for imaging a stent comprising the steps of
a. obtaining digital data representative of an image of a stent with a magnetic resonance imager wherein;
i. said stent is coated with a layer comprised of particulates wherein
1. said particulates have an average particle size of less than about 100 nanometers; and
2. said layer has a saturation magnetization of at least about 2,000 gauss.

37. The process for imaging a stent as recited in claim 36 further comprising the step of subjecting said digital data to an image post-processing method.

38. The process for imaging a stent as recited in claim 37 wherein said image post-processing method is a phase data post-processing method.

39. The process for imaging a stent as recited in claim 38 wherein said phase data post-processing method is phase equalization.

40. The process for imaging a stent as recited in claim 38 wherein said phase data post-processing method is phase edge detection.

41. The coated substrate as recited in claim 36, wherein said stent is selected from the group consisting of a nitinol stent and a copper stent.

42. The process for imaging a stent as recited in claim 41 wherein said stent is disposed within a biological organism.

43. The process for imaging a stent as recited in claim 42 wherein said biological organism is a human.

44. The process for imaging a stent as recited in claim 36 wherein said digital data is comprised of phase data.
